(19) United States
(12) Patent Application Publication
(10) Pub. No.: US 2024/0221913 A1
(43) Pub. Date: Jul. 4, 2024

(54) CHAT BOT FOR A MEDICAL IMAGING SYSTEM

(71) Applicant: KONINKLIJKE PHILIPS N.V., EINDHOVEN (NL)

(72) Inventors: Earl M. Canfield, II, New Braunfels, TX (US); Robert G. Trahms, Edmonds, WA (US)

(21) Appl. No.: 18/288,206

(22) PCT Filed: Apr. 25, 2022
(86) PCT No.: PCT/EP2022/060878

§ 371 Date: Oct. 25, 2023

Related U.S. Application Data
(60) Provisional application No. 63/180,920, filed on Apr. 28, 2021.

(51) Int. Cl. G16H 30/40; G06F 3/04812; G06F 40/20; G16H 10/60; G16H 30/20; H04L 51/02

(52) U.S. Cl. CPC G16H 30/40; G06F 3/04812; G06F 40/20; G16H 10/60; G16H 30/20; H04L 51/02

(57) ABSTRACT

An ultrasound imaging system may include a deep learning neural net chat bot feature that allows the user to interact with the system with natural language via text or voice to provide assistance while operating the system. The user can interact with the chat bot to receive natural language answers to questions involving system operation, configuration assistance, clinical assistance, training, marketing, and field service.

### TECHNICAL FIELD

The present disclosure pertains to a chat bot for a medical imaging system such as an ultrasound imaging system.

### BACKGROUND

A chat bot is a software application used to conduct a conversation with a user via text or speech, instead of interacting with a live human. Chat bot applications have existed for many years providing assistance using a pop-up text window on product websites to help users with purchasing decisions or resolve trivial problems. Until recently, most of these chat bot applications provided robotic and repetitive responses resulting in frustrated users requesting to speak to a live human to resolve issues. With technology advancements in artificial intelligence (AI), including neural networks, chat bots have become much better at understanding the user's intents (e.g., questions, requests) and produce conversations that mimic humans. Neural net chat bots interpret the user's intent by parsing typed or spoken words using large word classification processes to extract the essential key words and meanings. This data is then used to train a neural net model which results in an algorithm that can infer the user's intent from a wide variety of word combinations and dialects. An example of an AI-based chat bot is described in U.S. Patent Publication 2020/0143265, which is incorporated herein by reference for any purpose.

### SUMMARY

Systems and methods are disclosed that implement a chat bot on an ultrasound imaging system and/or in conjunction with an ultrasound imaging system. The chat bot may have access to information relating to the ultrasound imaging system's configuration and/or resources. The chat bot may communicate with one or more other applications on the ultrasound imaging system, including other AI applications (e.g., machine learning models). For example, an AI model for recognition of anatomical features in ultrasound images may communicate with the chat bot, which may allow the chat bot to provide information to a user regarding images provided on a display of the ultrasound imaging system. In some examples, the chat bot may be implemented, at least in part, on another device, such as a smart phone, in communication with the ultrasound imaging system. In some examples, this may permit a user to interact remotely with the ultrasound imaging system.

According to at least one example of the present disclosure, a medical imaging system may be configured to allow a user to interact with the medical imaging system via a chat bot and may include a user interface configured to receive a natural language user input, a non-transitory computer readable medium encoded with instructions to implement the chat bot and configured to store data related to the medical imaging system, and at least one processor in communication with the non-transitory computer readable medium configured to execute the instructions to implement the chat bot, wherein the instructions cause the at least one processor to determine an intent of the natural language user input, responsive to the intent, retrieve at least a portion of the data stored in the non-transitory computer readable medium or issue a command to be executed by the medical imaging system, and provide a natural language response to the user interface based, at least in part, on the portion of the data or the command.

According to at least one example of the present disclosure, a method for interacting with a medical imaging system with a chat bot may include receiving, via a user interface, a natural language user input, determining, with at least one processor configured to implement the chat bot, an intent of the natural language user input, responsive to the intent, retrieving data related to the medical imaging system stored on a non-transitory computer readable medium or issuing a command to be executed by the medical imaging system, and providing a natural language response to the user interface based, at least in part, the data or the command.

### BRIEF DESCRIPTION OF THE DRAWINGS

FIG. 1 is a block diagram of an ultrasound system in accordance with principles of the present disclosure.

FIG. 2 is a block diagram illustrating an example processor in accordance with principles of the present disclosure.

FIG. 3 is a diagram that provides an overview of examples of different applications of a chat bot on a medical imaging system according to principles of the present disclosure.

FIG. 4 illustrates an example of accessing a chat bot according to principles of the present disclosure.

FIG. 5 is an example text interaction between a chat bot and a user according to principles of the present disclosure.

FIG. 6 illustrates an example of a user interaction with a chat bot according to principles of the present disclosure.

FIG. 7 illustrates an example of a user interaction with a chat bot according to principles of the present disclosure.

FIG. 8 is a functional block diagram of a chat bot on an ultrasound imaging machine in accordance with principles of the present disclosure.

FIG. 9 is an illustration of a neural network that may be used to analyze user intents in accordance with examples of the present disclosure.

FIG. 10 is a block diagram of a process for training and deployment of a neural network in accordance with the principles of the present disclosure.

FIG. 11 is a flow chart of a method in accordance with principles of the present disclosure.

### DETAILED DESCRIPTION

The following description of certain embodiments is merely exemplary in nature and is in no way intended to limit the invention or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the present system. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present system is defined only by the appended claims.

When users have questions or problems with an ultrasound imaging system, they currently have two options: 1) Find a help option in a menu to access the help files and search for a relevant topic: or 2) call tech support. Searching the help documentation can answer many user questions, but users must manually search for relevant information via normal search screens and indexes—this can be tedious and takes the user's valuable time. Calling tech support may resolve most issues, but users must call and wait for a technical or field service person to respond, which is also time consuming, even when the answer the user is looking for may be quite simple in nature. Furthermore, neither option may be feasible if an issue arises during an exam.

With a chat bot feature on the ultrasound system, users can quickly interact via text and/or speech and get an immediate response via text and/or speech. Using a chat bot interface, users can ask specific questions with a single response similar to a question/answer FAQ format or users can ask questions which results in a diagnostic tree response where the chat bot responds with further questions to isolate and troubleshoot the problem. Users can also receive links to relevant system or training material available on numerous sites maintained by the manufacturer of the ultrasound imaging system. The content links displayed may be based, at least in part, on what the chat bot interprets the user's intent and interest in particular subject content is.

According to examples of the present disclosure, a chat bot may have “knowledge” (e.g., access to data/information relating to) of the user's ultrasound system model, purchased options, hardware, resources, configurations, and/or other features. Users can interact with the chat bot using natural language (e.g., language developed naturally for human use rather than computer code) about specific issues on their system such as wireless/network connection problems. IP addresses, exam export status, specific configuration questions, and/or other issues. According to examples of the present disclosure, the chat bot may further have knowledge of what the user is currently doing on the ultrasound imaging system (e.g., the exam type selected, current acquisition settings) and/or what the user is viewing on the screen (e.g., an ultrasound image of a 4-chamber view of the heart). Thus, in some examples, the chat bot may answer specific questions about exam types or an image currently acquired by the ultrasound imaging system.

In some examples, there can be multiple chat bot knowledge bases (e.g., databases) with specific knowledge to answer questions in a particular area of focus. In some examples, other applications on the ultrasound imaging system may communicate with the chat bot to provide the knowledge to answer a user's inquiries. For example, an application (e.g., a machine learning model) configured to identify anatomical features in an ultrasound image may provide information on any identified anatomical features. In some examples, on the ultrasound system a typical text window may be displayed in the corner of the screen, when users hovers over the area, the chat bot responds with a greeting. In the diagram below the chat bot is referred to as “Philippa” or other Philips marketing name. Once the user types (or speaks) a question in natural language, the phrase may be parsed and interpreted by a machine learning model and passed to the appropriate application, which may be another machine learning model, to determine the response. In some examples, at any time, the user can request to speak to a live support person for further assistance.

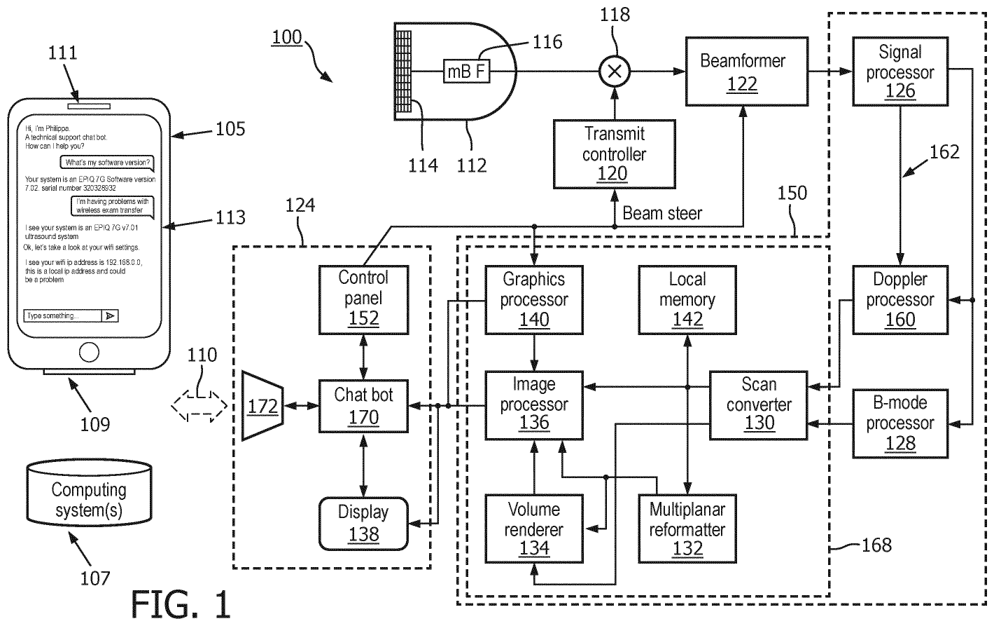

FIG. 1 shows a block diagram of an ultrasound imaging system 100 constructed in accordance with the principles of the present disclosure. An ultrasound imaging system 100 according to the present disclosure may include a transducer array 114, which may be included in an ultrasound probe 112, for example an external probe or an internal probe such as an Intra Cardiac Echography (ICE) probe or a Trans Esophagus Echography (TEE) probe. In other embodiments, the transducer array 114 may be in the form of a flexible array configured to be conformably applied to a surface of subject to be imaged (e.g., patient). The transducer array 114 is configured to transmit ultrasound signals (e.g., beams, waves) and receive echoes responsive to the ultrasound signals. A variety of transducer arrays may be used. e.g., linear arrays, curved arrays, or phased arrays. The transducer array 114, for example, can include a two dimensional array (as shown) of transducer elements capable of scanning in both elevation and azimuth dimensions for 2D and/or 3D imaging. As is generally known, the axial direction is the direction normal to the face of the array (in the case of a curved array the axial directions fan out), the azimuthal direction is defined generally by the longitudinal dimension of the array, and the elevation direction is transverse to the azimuthal direction.

In some embodiments, the transducer array 114 may be coupled to a microbeamformer 116, which may be located in the ultrasound probe 112, and which may control the transmission and reception of signals by the transducer elements in the array 114. In some embodiments, the microbeamformer 116 may control the transmission and reception of signals by active elements in the array 114 (e.g., an active subset of elements of the array that define the active aperture at any given time). In some embodiments, the microbeamformer 116 may be coupled. e.g., by a probe cable or wirelessly, to a transmit/receive (T/R) switch 118, which switches between transmission and reception and protects a main beamformer 122 from high energy transmit signals. In some embodiments, for example in portable ultrasound systems, the T/R switch 118 and other elements in the system can be included in the ultrasound probe 112 rather than in the ultrasound system base, which may house the image processing electronics. An ultrasound system base typically includes software and hardware components including circuitry for signal processing and image data generation as well as executable instructions for providing a user interface (e.g., processing circuitry 150 and user interface 124).

The transmission of ultrasonic signals from the transducer array 114 under control of the microbeamformer 116 is directed by the transmit controller 120, which may be coupled to the T/R switch 218 and the main beamformer 122. The transmit controller 120 may control the direction in which beams are steered. Beams may be steered straight ahead from (orthogonal to) the transducer array 114, or at different angles for a wider field of view. The transmit controller 120 may also be coupled to a user interface 124 and receive input from the user's operation of a user control. The user interface 124 may include one or more input devices such as a control panel 152, which may include one or more mechanical controls (e.g., buttons, encoders, etc.), touch sensitive controls (e.g., a trackpad, a touchscreen, or the like), and/or other known input devices.

In some embodiments, the partially beamformed signals produced by the microbeamformer 116 may be coupled to a main beamformer 122 where partially beamformed signals from individual patches of transducer elements may be combined into a fully beamformed signal. In some embodiments, microbeamformer 116 is omitted, and the transducer array 114 is under the control of the main beamformer 122 which performs all beamforming of signals. In embodiments with and without the microbeamformer 116, the beamformed signals of the main beamformer 122 are coupled to processing circuitry 150, which may include one or more processors (e.g., a signal processor 126, a B-mode processor 128, a Doppler processor 160, and one or more image generation and processing components 168) configured to produce an ultrasound image from the beamformed signals (e.g., beamformed RF data).

The signal processor 126 may be configured to process the received beamformed RF data in various ways, such as bandpass filtering, decimation. I and Q component separation, and harmonic signal separation. The signal processor 126 may also perform additional signal enhancement such as speckle reduction, signal compounding, and noise elimination. The processed signals (also referred to as I and Q components or IQ signals) may be coupled to additional downstream signal processing circuits for image generation. The IQ signals may be coupled to a plurality of signal paths within the system, each of which may be associated with a specific arrangement of signal processing components suitable for generating different types of image data (e.g., B-mode image data. Doppler image data). For example, the system may include a B-mode signal path 158 which couples the signals from the signal processor 126 to a B-mode processor 128 for producing B-mode image data.

The B-mode processor can employ amplitude detection for the imaging of structures in the body. The signals produced by the B-mode processor 128 may be coupled to a scan converter 130 and/or a multiplanar reformatter 132. The scan converter 130 may be configured to arrange the echo signals from the spatial relationship in which they were received to a desired image format. For instance, the scan converter 130 may arrange the echo signal into a two dimensional (2D) sector-shaped format, or a pyramidal or otherwise shaped three dimensional (3D) format. The multiplanar reformatter 132 can convert echoes which are received from points in a common plane in a volumetric region of the body into an ultrasonic image (e.g., a B-mode image) of that plane, for example as described in U.S. Pat. No. 6,443,896 (Detmer). The scan converter 130 and multiplanar reformatter 132 may be implemented as one or more processors in some embodiments.

A volume renderer 134 may generate an image (also referred to as a projection, render, or rendering) of the 3D dataset as viewed from a given reference point. e.g., as described in U.S. Pat. No. 6,530,885 (Entrekin et al.). The volume renderer 134 may be implemented as one or more processors in some embodiments. The volume renderer 134 may generate a render, such as a positive render or a negative render, by any known or future known technique such as surface rendering and maximum intensity rendering.

In some embodiments, the system may include a Doppler signal path 162 which couples the output from the signal processor 126 to a Doppler processor 160. The Doppler processor 160 may be configured to estimate the Doppler shift and generate Doppler image data. The Doppler image data may include color data which is then overlaid with B-mode (i.e. grayscale) image data for display. The Doppler processor 160 may be configured to filter out unwanted signals (i.e., noise or clutter associated with non-moving tissue), for example using a wall filter. The Doppler processor 160 may be further configured to estimate velocity and power in accordance with known techniques. For example, the Doppler processor may include a Doppler estimator such as an auto-correlator, in which velocity (Doppler frequency) estimation is based on the argument of the lag-one autocorrelation function and Doppler power estimation is based on the magnitude of the lag-zero autocorrelation function. Motion can also be estimated by known phase-domain (for example, parametric frequency estimators such as MUSIC. ESPRIT, etc.) or time-domain (for example, cross-correlation) signal processing techniques. Other estimators related to the temporal or spatial distributions of velocity such as estimators of acceleration or temporal and/or spatial velocity derivatives can be used instead of or in addition to velocity estimators. In some embodiments, the velocity and/or power estimates may undergo further threshold detection to further reduce noise, as well as segmentation and post-processing such as filling and smoothing. The velocity and/or power estimates may then be mapped to a desired range of display colors in accordance with a color map. The color data, also referred to as Doppler image data, may then be coupled to the scan converter 130, where the Doppler image data may be converted to the desired image format and overlaid on the B-mode image of the tissue structure to form a color Doppler or a power Doppler image. In some examples, the scan converter 130 may align the Doppler image and B-mode image

Outputs from the scan converter 130, the multiplanar reformatter 132, and/or the volume renderer 134 may be coupled to an image processor 136 for further enhancement, buffering and temporary storage before being displayed on an image display 138. A graphics processor 140 may generate graphic overlays for display with the images. These graphic overlays can contain. e.g., standard identifying information such as patient name, date and time of the image, imaging parameters, and the like. For these purposes the graphics processor may be configured to receive input from the user interface 124, such as a typed patient name or other annotations. The user interface 124 can also be coupled to the multiplanar reformatter 132 for selection and control of a display of multiple multiplanar reformatted (MPR) images.

The ultrasound imaging system 100 may include local memory 142. Local memory 142 may be implemented as any suitable non-transitory computer readable medium (e.g., flash drive, disk drive). Local memory 142 may store data generated by the ultrasound imaging system 100 including ultrasound images, executable instructions, training data sets, and/or any other information necessary for the operation of the ultrasound imaging system 100. Although not all connections are shown to avoid obfuscation of FIG. 1, local memory 142 may be accessible by additional components other than the scan converter 130, multiplanar reformatter 132, and image processor 136. For example, the local memory 142 may be accessible to the graphics processor 140, transmit controller 120, signal processor 126, user interface 124, etc.

As mentioned previously ultrasound imaging system 100 includes user interface 124. User interface 124 may include display 138 and control panel 152. The display 138 may include a display device implemented using a variety of known display technologies, such as LCD. LED. OLED, or plasma display technology. In some embodiments, display 138 may comprise multiple displays. The control panel 152 may be configured to receive user inputs (e.g., pre-set number of frames, exam type, imaging mode). The control panel 152 may include one or more hard controls (e.g., microphone/speaker, buttons, knobs, dials, encoders, mouse, trackball or others). Hard controls may sometimes be referred to as mechanical controls. In some embodiments, the control panel 152 may additionally or alternatively include soft controls (e.g., GUI control elements, or simply GUI controls such as buttons and sliders) provided on a touch sensitive display. In some embodiments, display 138 may be a touch sensitive display that includes one or more soft controls of the control panel 152.

In some embodiments, various components shown in FIG. 1 may be combined. For instance, in some examples, a single processor may implement multiple components of the processing circuitry 150 (e.g., image processor 136, graphics processor 140) as well as the chat bot 170. In some embodiments, various components shown in FIG. 1 may be implemented as separate components. For example, signal processor 126 may be implemented as separate signal processors for each imaging mode (e.g., B-mode. Doppler. SWE). In some embodiments, one or more of the various processors shown in FIG. 1 may be implemented by general purpose processors and/or microprocessors configured to perform the specified tasks. In some embodiments, one or more of the various processors may be implemented as application specific circuits. In some embodiments, one or more of the various processors (e.g., image processor 136) may be implemented with one or more graphical processing units (GPU).

According to examples of the present disclosure, ultrasound imaging system 100 may include a chat bot 170 that may interact with a user using natural language via the user interface 124. For example, the user interface 124 may have a dedicated soft or hard control for activating the chat bot 170. In some examples, a text window or icon may be provided in a corner of display 138. When a user clicks, taps, and/or hovers over the area, the chat bot 170 may respond with a greeting and/or the icon may expand to show a window where the user can enter text. Once the user types a natural language input (e.g., question or command), the input phrase may be parsed and interpreted to determine what the user has requested in the input (e.g., intent). The processed phrase may be provided to one or more neural networks to determine an output (e.g., natural language response and/or action) to the user's input. Additionally or alternatively, the user may interact with the chat bot 170 by voice. For example, the chat bot 170 may “listen” for an activation phrase, such as “Hey. Philippa” rather than waiting for the user to click, tap, or hover over an icon. The user may then provide the input orally after saying the activation phrase.

In some examples, the chat bot 170 may include one or more processors and/or be implemented by execution of computer readable instructions (e.g., such as computer readable instructions stored on local memory 142) by one or more processors and/or application specific integrated circuits. The chat bot 170 may respond to user inputs provided by a user. In some examples, the chat bot 170 may receive inputs via a keyboard and/or touch screen included in the user interface 124. In some examples, the chat bot 170 may receive inputs from the user via a microphone. The inputs from the user may be questions and/or requests. In some examples, the chat bot 170 may provide a natural language output to the user via display 138 and/or a speaker/microphone 172 included with user interface 124. The output may be an answer to a question, fulfillment of a request, and/or a conformation that a request has been fulfilled.

Although the connections are not shown to avoid obfuscating FIG. 1, chat bot 170 may be capable of receiving information from and/or providing instructions to one or more components of ultrasound imaging system 100. For example, chat bot 170 may receive instructions and/or data from local memory 142. In another example, chat bot 170 may provide instructions to the transmit controller 120 based on a user input. In a further example, chat bot 170 may receive information relating to an ultrasound image provided on display 138 from image processor 136. Although shown within the user interface 124 in FIG. 1, the chat bot 170 need not be physically located within or immediately adjacent to the user interface 124. For example, chat bot 170 may be located with processing circuitry 150.

In some examples, the chat bot 170 may include and/or implement any one or more machine learning models, deep learning models, artificial intelligence algorithms, and/or neural networks (collectively, models) which may analyze the natural language user input to determine the user's intent. In some examples, chat bot 170 may include a long short term (LSTM) model, deep neural network (DNN), a convolutional neural network (CNN), a recurrent neural network (RNN), an autoencoder neural network, or the like, to determine the user's intent (e.g., question, request). The model and/or neural network may be implemented in hardware (e.g., neurons are represented by physical components) and/or software (e.g., neurons and pathways implemented in a software application) components. The model and/or neural network implemented according to the present disclosure may use a variety of topologies and learning algorithms for training the model and/or neural network to produce the desired output. For example, a software-based neural network may be implemented using a processor (e.g., single or multi-core CPU, a single GPU or GPU cluster, or multiple processors arranged for parallel-processing) configured to execute instructions, which may be stored in computer readable medium, and which when executed cause the processor to perform a trained algorithm for determining a user's intent and responding thereto (e.g., receiving a question and providing the appropriate answer to the question). In some embodiments, the chat bot 170 may implement a model and/or neural network in combination with other data processing methods (e.g., statistical analysis).

In various embodiments, the model(s) may be trained using any of a variety of currently known or later developed learning techniques to obtain a model (e.g., a trained algorithm, transfer function, or hardware-based system of nodes) that is configured to analyze user inputs (e.g., sentences and portions thereof, whether typed or spoken). In some embodiments, the model may be statically trained. That is, the model may be trained with a data set and deployed on the chat bot 170. In some embodiments, the model may be dynamically trained. In these embodiments, the model may be trained with an initial data set and deployed on the ultrasound system 100. However, the model may continue to train and be modified based on inputs acquired by the chat bot 170 after deployment of the model on the ultrasound imaging system 100.

Optionally, in some examples, the ultrasound imaging system 100 may be in communication with one or more devices via one or more communication channels 110. The communication channels 110 may be wired (e.g., Ethernet. USB) or wireless (e.g., Bluetooth. Wi-Fi). In some examples, the ultrasound imaging system 100 may communicate with one or more computing systems 107. The computing systems 107 may include hospital servers, which may include electronic medical records of patients. The medical records may include images from previous exams. The computing systems 107 may include a picture archiving computer system (PACS). In some examples, the chat bot 170 may interact with the computing systems 107 (or another application on ultrasound imaging system 100 that interacts with the computing systems 107) via the communication channel 110 to respond to a user input.

In some examples, the ultrasound imaging system 100 may communicate with a mobile device 105, such as a smart phone, tablet, and/or laptop. In some examples, the mobile device 105 may include an application that implements some of the chat bot 170. For example, the mobile device 105 may include an application that permits the chat bot 170 to utilize the speaker 109, microphone 111, and/or display 113 (which may be a touch screen) of the mobile device 105 to receive natural language inputs and/or provide natural language outputs. In some examples, the mobile device 105 may provide received user inputs to the ultrasound imaging system 100, and the ultrasound imaging system 100 may provide responses to the inputs to the mobile device 105. In other words, the mobile device 105 may be an extension of the user interface 124. In another example, the mobile device 105 may include an application that permits the chat bot 170 to communicate with remotely located servers and/or other resources (e.g., transmit data to and/or call the technical support department of the manufacturer of the ultrasound imaging system 100).

In some embodiments, various components shown in FIG. 1 may be combined. For instance, in some examples, a single processor may implement multiple components of the processing circuitry 150 (e.g., image processor 136, graphics processor 140) as well as the chat bot 170. In some embodiments, various components shown in FIG. 1 may be implemented as separate components. For example, signal processor 126 may be implemented as separate signal processors for each imaging mode (e.g., B-mode. Doppler. SWE). In some embodiments, one or more of the various processors shown in FIG. 1 may be implemented by general purpose processors and/or microprocessors configured to perform the specified tasks. In some embodiments, one or more of the various processors may be implemented as application specific circuits. In some embodiments, one or more of the various processors (e.g., image processor 136) may be implemented with one or more graphical processing units (GPU).

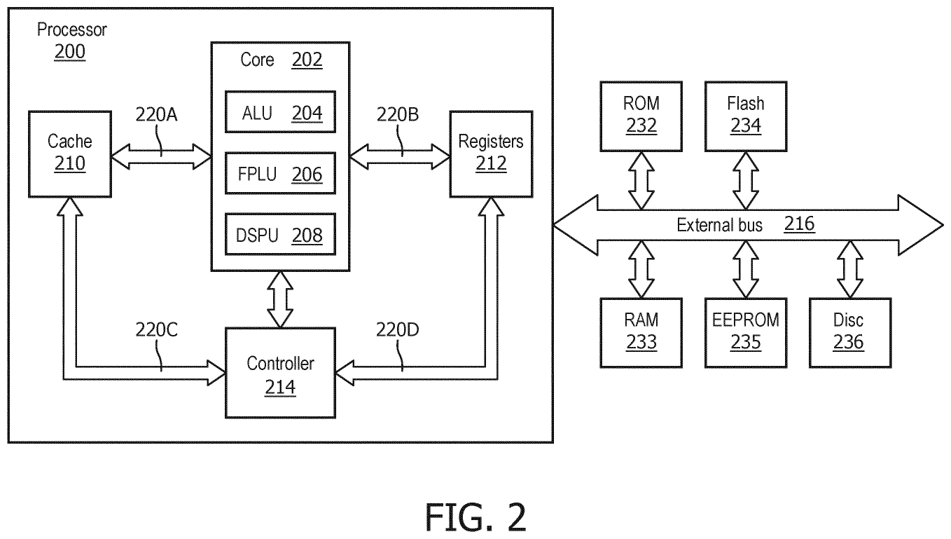

FIG. 2 is a block diagram illustrating an example processor 200 according to principles of the present disclosure. Processor 200 may be used to implement one or more processors and/or controllers described herein, for example, image processor 136, graphics processor 140, and/or one or more processors implementing the chat bot 170 and/or any other processor or controller shown in FIG. 1. Processor 200 may be any suitable processor type including, but not limited to, a microprocessor, a microcontroller, a digital signal processor (DSP), a field programmable array (FPGA) where the FPGA has been programmed to form a processor, a graphical processing unit (GPU), an application specific circuit (ASIC) where the ASIC has been designed to form a processor, or a combination thereof. The processor 200 may include one or more cores 202. The core 202 may include one or more arithmetic logic units (ALU) 204. In some embodiments, the core 202 may include a floating point logic unit (FPLU) 206 and/or a digital signal processing unit (DSPU) 208 in addition to or instead of the ALU 204.

The processor 200 may include one or more registers 212 communicatively coupled to the core 202. The registers 212 may be implemented using dedicated logic gate circuits (e.g., flip-flops) and/or any memory technology. In some embodiments the registers 212 may be implemented using static memory. The register may provide data, instructions and addresses to the core 202.

In some embodiments, processor 200 may include one or more levels of cache memory 210 communicatively coupled to the core 202. The cache memory 210 may provide computer-readable instructions to the core 202 for execution. The cache memory 210 may provide data for processing by the core 202. In some embodiments, the computer-readable instructions may have been provided to the cache memory 210 by a local memory, for example, local memory attached to the external bus 216. The cache memory 210 may be implemented with any suitable cache memory type, for example, metal-oxide semiconductor (MOS) memory such as static random access memory (SRAM), dynamic random access memory (DRAM), and/or any other suitable memory technology.

The processor 200 may include a controller 214, which may control input to the processor 200 from other processors and/or components included in a system (e.g., control panel 152 and scan converter 130 shown in FIG. 1) and/or outputs from the processor 200 to other processors and/or components included in the system (e.g., display 138 and volume renderer 134 shown in FIG. 1). Controller 214 may control the data paths in the ALU 204. FPLU 206 and/or DSPU 208. Controller 214 may be implemented as one or more state machines, data paths and/or dedicated control logic. The gates of controller 214 may be implemented as standalone gates. FPGA. ASIC or any other suitable technology.

The registers 212 and the cache memory 210 may communicate with controller 214 and core 202 via internal connections 220A, 220B. 220C and 220D. Internal connections may implemented as a bus, multiplexor, crossbar switch, and/or any other suitable connection technology.

Inputs and outputs for the processor 200 may be provided via a bus 216, which may include one or more conductive lines. The bus 216 may be communicatively coupled to one or more components of processor 200, for example the controller 214, cache memory 210, and/or register 212. The bus 216 may be coupled to one or more components of the system, such as display 138 and control panel 152 mentioned previously.

The bus 216 may be coupled to one or more external memories. The external memories may include Read Only Memory (ROM) 232. ROM 232 may be a masked ROM. Electronically Programmable Read Only Memory (EPROM) or any other suitable technology. The external memory may include Random Access Memory (RAM) 233. RAM 233 may be a static RAM, battery backed up static RAM. Dynamic RAM (DRAM) or any other suitable technology. The external memory may include Electrically Erasable Programmable Read Only Memory (EEPROM) 235. The external memory may include Flash memory 234. The external memory may include a magnetic storage device such as disc 236. In some embodiments, the external memories may be included in a system, such as ultrasound imaging system 100 shown in FIG. 1, for example local memory 142.

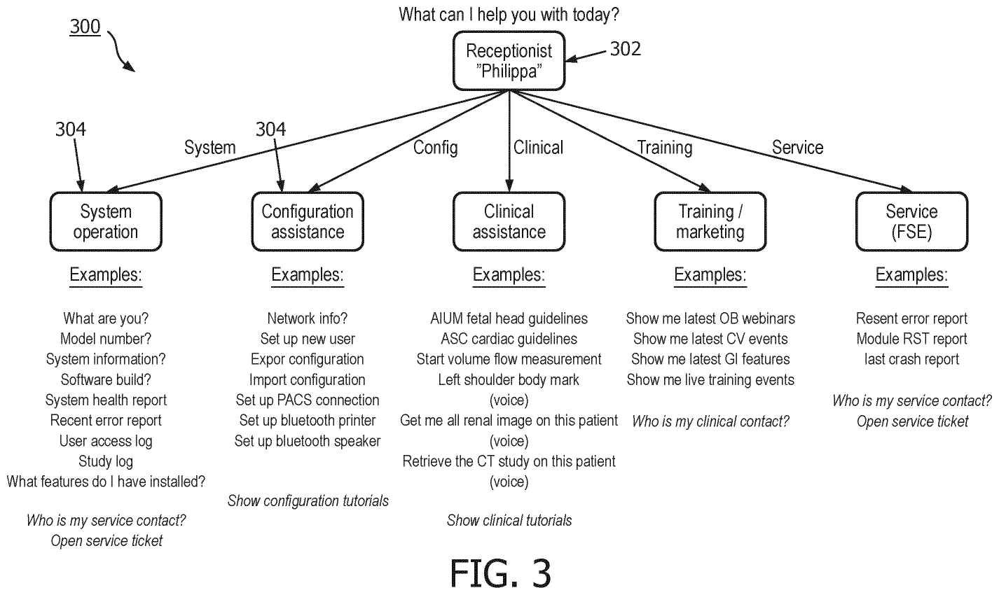

FIG. 3 is a diagram that provides an overview of examples of different applications of a chat bot on a medical imaging system according to principles of the present disclosure. As shown in diagram 300, a chat bot 302 on a medical imaging system, such as chat bot 170 on ultrasound imaging system 100, may act as a “receptionist” that can direct users to different resources and/or access different resources to assist the user using natural language. In some examples, there may be multiple chat bot knowledge bases 304, each with specific knowledge to answer questions in a particular area of focus. In the example shown, the areas include system operation, configuration assistance, clinical assistance, training/marketing, and service. However, fewer, additional and/or different knowledge bases may be included in other examples. The knowledge bases 304 may include data included in files, databases, and/or passed from another application (e.g., an anatomical feature identification machine learning model, a measurement tool set). In some examples, some or all of the data may be stored in non-transitory computer-readable media, such as local memory 142, which is accessible to the chat bot 302.

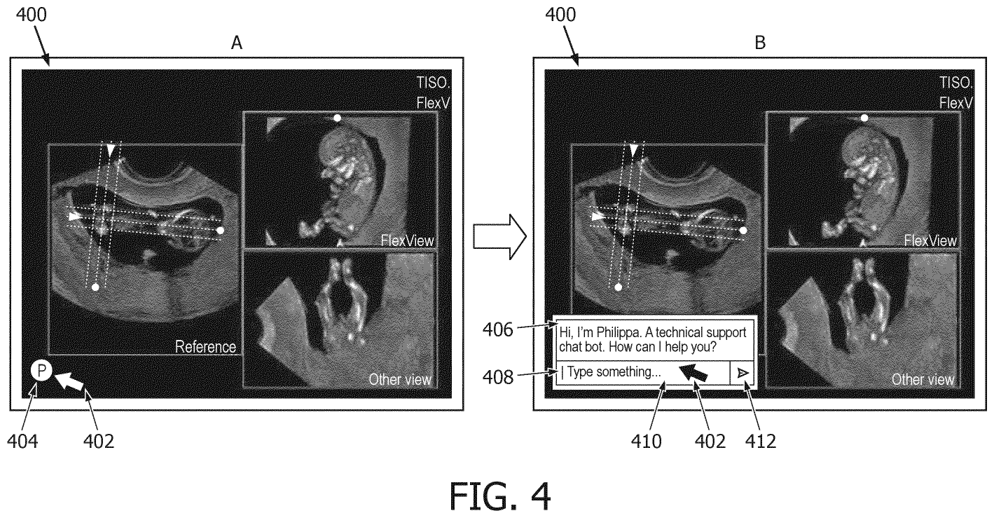

FIG. 4 illustrates an example of accessing a chat bot according to principles of the present disclosure. Display 400 may be included in an ultrasound imaging system in some examples, such as ultrasound imaging system 100. In some examples, display 400 may be included in display 138. In some examples, display 400 may provide various GUI elements such as a cursor 402 and selectable icons, such as chat bot icon 404. The user may move the cursor 402 using a trackball, arrow keys, mouse, touchpad, joystick, and/or any other suitable technique. The user may use the cursor 402 to interact with the ultrasound imaging system. For example, the user may access measurement tools to measure features in an ultrasound image. In another example, the user may access a chat bot, such as chat bot 170, by moving the cursor 402 to the chat bot icon 404 as shown in panel A. When a user clicks, taps, and/or hovers the cursor 402 over the chat bot icon 404, as shown in panel B, the chat bot icon 404 may expand into a dialog box 406. The dialog box 406 may include a greeting 408, a text box 410 where a user can enter text, and/or a send icon 412 that allows the user to submit any entered text to the chat bot for processing. In some examples, the dialog box 406 may include additional features, such as an icon (not shown) that allows the user to provide inputs to the chat bot via speech rather than text.

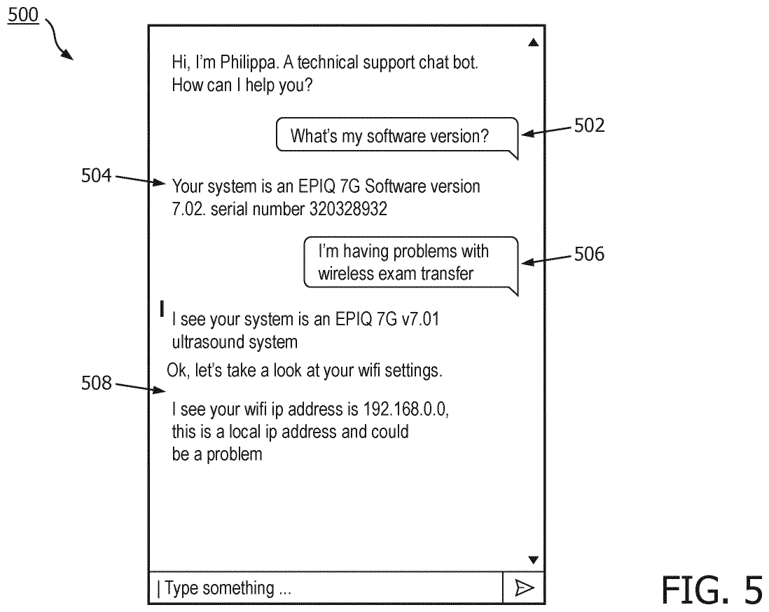

FIG. 5 is an example text interaction between a chat bot and a user according to principles of the present disclosure. The dialog box 500 may be provided on a display, such as display 138 and/or display 400 in some examples. The dialog box 500 may implement dialog box 406 in some examples. The dialog box 500 may have been provided responsive to a user clicking on a chat bot icon, for example, as described with reference to FIG. 4. Alternatively, the dialog box 500 may have been provided responsive to an oral command (e.g., “Hey. Phillipa”) issued by the user.

In the example shown, the user has input an initial inquiry 502. “What's my software version?” The chat bot provides a response 504. “Your system is an EPIQ 7G. Software Version 7.02. Serial Number 320328923.” In some examples, a trained machine learning model, such as a neural network, of the chat bot may have analyzed the natural language of the initial inquiry 502 to infer the user's intent (e.g., wanting to know the software version). Based on the intent output by the machine learning model, the chat bot may retrieve (e.g., send a query to the appropriate component of the ultrasound imaging system, such as the local memory, and receive a response from the component to the query) the appropriate information (e.g., software version information) from the ultrasound imaging system (e.g., information from a database, file, or other data structure stored on local memory 142) to provide the response 504.

The user need not provide an input in the form of a question. As shown in FIG. 5, a second input 506 states. “I'm having problems with wireless exam transfer.” The chat bot analyzes the user's input and infers the user is having trouble with WI-FI. The chat bot then retrieves the current WI-FI settings and indicates that the WI-FI settings may be incorrect as indicated by response 508. “I see your wifi ip address is 192.168.0.0, this is a local ip address and could be a problem.” In some examples, the determination that the IP address is local and may be a problem for wireless exam transfer may not be determined by the chat bot. Rather, another application on the ultrasound imaging system (e.g., ultrasound imaging system 100) for controlling wireless communications may provide the information and determination responsive to a query by the chat bot.

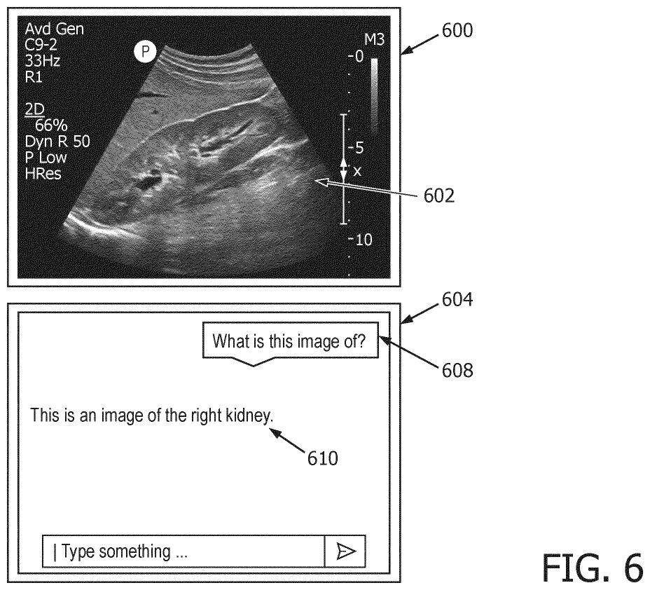

FIG. 6 illustrates an example of a user interaction with a chat bot according to principles of the present disclosure. Display 600 may be included in an ultrasound imaging system in some examples, such as ultrasound imaging system 100. In some examples, display 600 may be included in display 138. In some examples, display 600 may provide ultrasound image 602 acquired by an ultrasound probe such as ultrasound probe 112. In some examples, an ultrasound imaging system including display 600 may additionally include display 604. In some examples, display 604 may provide various GUI elements, such as a dialog box 606 for a chat bot, such as chat bot 170. In some examples, display 604 may be a touch screen. In some examples, display 604 may be smaller than display 600. Of course, in other examples, displays 600 and 604 may be the same size or display 604 may be larger than display 600. In some examples, both displays 600 and 604 may be touch screens. In some examples, both displays 600 and 604 may provide ultrasound images and GUI elements. In some examples, display 604 may be a display of a mobile device in communication with the ultrasound imaging system, such as mobile device 105.

In the example shown in FIG. 6, a user provides a natural language input 608 inquiring about ultrasound image 602. The chat bot provides a response 610 indicating that image 602 includes the right kidney of a subject. The ultrasound image 602 may be an image displayed on display 600 during review (e.g., retrieved from an image file after an exam) or the ultrasound image 602 may be “live.” that is, just acquired and/or currently being acquired by an ultrasound probe during an exam. In some examples, such as when the ultrasound image 602 is being reviewed by the user after an exam, the chat bot may query the image file for labels and/or annotations to determine what is included in ultrasound image 602. The chat bot may use the labels and/or annotations to provide the response 610. In some examples, such as when the ultrasound image 602 is live and/or being reviewed, the chat bot may query a machine learning model trained to identify anatomical features in the ultrasound image 602. The machine learning model may analyze the ultrasound image 602 and provide an inference as to the anatomical feature(s) present in the ultrasound image 602 to the chat bot. The chat bot may use the inference to provide the response 610. Thus, the chat bot may be “aware” of what the user is viewing on display 600.

In the examples shown in FIGS. 4-6, the user initiates interactions with the chat bot. However, in other examples, the chat bot may be “aware” of what the user is doing and prompt the user to interact.

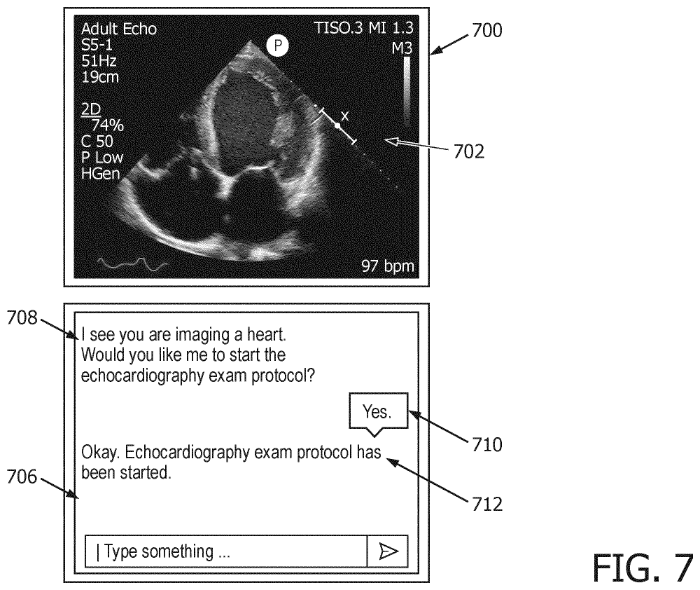

FIG. 7 illustrates an example of a user interaction with a chat bot according to principles of the present disclosure. Display 700 may be included in an ultrasound imaging system in some examples, such as ultrasound imaging system 100. In some examples, display 700 may be included in display 138. In some examples, display 700 may provide ultrasound image 702 acquired by an ultrasound probe such as ultrasound probe 112. In some examples, an ultrasound imaging system including display 700 may additionally include display 704. In some examples, display 704 may provide various GUI elements, such as a dialog box 706 for a chat bot, such as chat bot 170. In some examples, display 704 may be a touch screen. In some examples, display 704 may be smaller than display 700. Of course, in other examples, displays 700 and 704 may be the same size or display 704 may be larger than display 700. In some examples, both displays 700 and 704 may be touch screens. In some examples, both displays 700 and 704 may provide ultrasound images and GUI elements. In some examples, display 704 may be a display of a mobile device in communication with the ultrasound imaging system, such as mobile device 105.

In some examples, the chat bot may monitor activity on the ultrasound imaging system (e.g., user inputs, settings). The chat bot may provide a natural language prompt to a user responsive to certain actions taken by the user and/or ultrasound imaging system. In some examples, other applications on the ultrasound imaging system may trigger the chat bot to provide a prompt when the application receives a particular input from the user and/or another predetermined event occurs.

In the example shown in FIG. 7, an echocardiogram is being performed, and ultrasound image 702 includes a view of a heart of a subject. In some examples, the chat bot may receive (or request and receive) an indication as to an exam type selected by the user. In some examples, the chat bot may receive (or request and receive) a determination that ultrasound image 702 includes a view of the heart from a machine learning model. Based on the exam type and/or the inclusion of the heart in ultrasound image 702, the chat bot may provide a prompt 708 that offers assistance to the user particular to the exam type and/or anatomy being imaged. In the example shown, the chat bot offers to initiate a protocol for echocardiography exams. The user provides an input 710 accepting the offer of assistance. The chat bot may then send a command to the appropriate application on the ultrasound imaging system to initiate the assistance (e.g., initiate the echocardiography exam protocol). Once the command is sent, the chat bot may provide a confirmation 712 to the user. Other examples of assistance the chat bot may offer include, but are not limited to, providing appropriate measurement tools on the GUI, contacting tech support, and initiating a troubleshooting wizard.

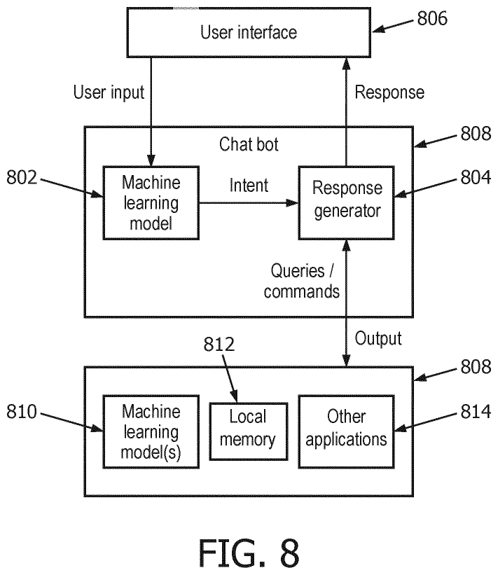

FIG. 8 is a functional block diagram of a chat bot on an ultrasound imaging machine in accordance with principles of the present disclosure. In some examples, chat bot 800 may be implemented by one or processors. In some examples, the one or more processors may implement the chat bot 800 by executing instructions provided by one or more non-transitory computer readable mediums. The chat bot 800 may be included in an ultrasound imaging system, such as ultrasound imaging system 100. In some examples, the chat bot 800 may include a machine learning model 802 and a response generator 804. In some examples, the machine learning model 802 and response generator 804 may be implemented by separate processors. In other examples, they may be implemented by a same processor or same group of processors. In some examples, chat bot 800 may be used to implement chat bot 170.

The machine learning model 802 may be training to infer user intents based on natural language user inputs received via a user interface 806. In some examples, user interface 806 may be included in user interface 124. In some examples, at least a portion of the user interface 806 may be included on a mobile device, such as mobile device 105. The intent determined by the machine learning model 802 may be provided to the response generator 804. The response generator 804 may generate a natural language response to provide to the user via the user interface 806. In some examples, the response generator 804 may provide one or more queries and/or commands based on the intent output by the machine learning model 802. The queries and/or commands may be provided to other components 808 of the ultrasound imaging system. In the example shown, the other components 808 may include other machine learning models 810, a local memory 812, and/or other applications 814 of the ultrasound imaging system.

For example, the response generator 804 may send a query to the local memory 812 when the intent indicates a user wants to retrieve a previous exam of a subject. In another example, the response generator 804 may query the machine learning model 810 when a user wants to know what anatomical feature is currently being displayed on a display of the ultrasound imaging system. In another example, the response generator 804 may send a command to other applications 814, such as when the user wants to change acquisition settings (e.g., increase gain, switch to Doppler imaging mode).

Based on the response to the query and/or a confirmation that a command has been implemented, the response generator 804 may provide a natural language response to the user's input to answer the user's question and/or confirm the user's request has been completed or initiated. Or, if the user's query cannot be answered or request cannot be completed, the response generator 804 will provide an indication of such (e.g., “I'm sorry. I cannot find an exam for Jane Doc.” “I'm sorry, that setting is not compatible with the probe you are using.”). As noted with reference to FIG. 7, in some examples, the response generator 804 may provide a prompt responsive to a trigger from one of the components 808 rather than responsive to an intent provided by the machine learning model 802.

In some embodiments, when the machine learning model 802 cannot infer the user's intent from the input, or a confidence level associated with the inference (e.g., a probability that the inference is correct) is below a threshold value, the response generator 804 may provide an indication of such (e.g., “I'm sorry. I don't understand.”). In other embodiments, the response generator 804 may prompt the user for more information when the confidence level is below the threshold value. For example, if the machine learning model 802 recognizes the user input refers to Bluetooth but cannot otherwise infer the user's intent, the response generator 804 may provide a prompt. “Do you want help connecting a Bluetooth device?”

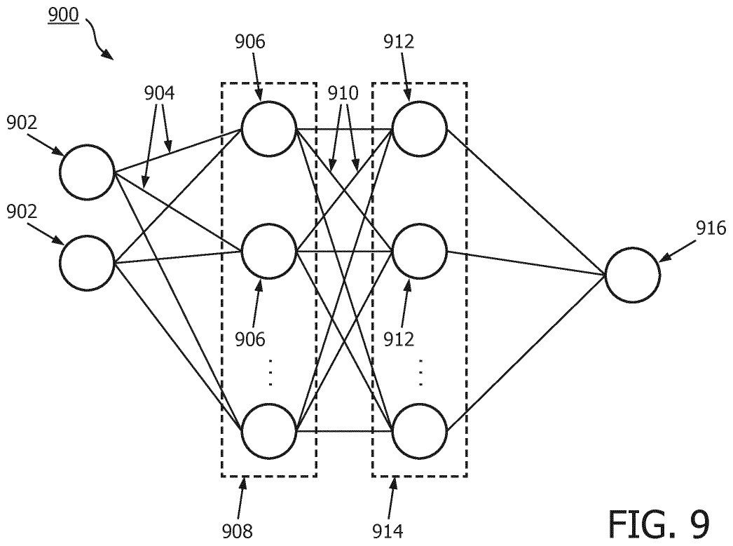

FIG. 9 is an illustration of a neural network that may be used to analyze user intents in accordance with principles of the present disclosure. In some examples, the neural network 900 may be implemented by one or more processors of an ultrasound imaging system (e.g., ultrasound imaging system 100) to implement a machine learning model (e.g., machine learning model 802). The machine learning model may be included in a chat bot, such as chat bot 170 and/or chat bot 800. In some examples, neural network 900 may be a convolutional network with single and/or multidimensional layers. The neural network 900 may include one or more input nodes 902. In some examples, the input nodes 902 may be organized in a layer of the neural network 900. The input nodes 902 may be coupled to one or more layers 908 of hidden units 906 by weights 904. In some examples, the hidden units 906 may perform operations on one or more inputs from the input nodes 902 based, at least in part, with the associated weights 904. In some examples, the hidden units 906 may be coupled to one or more layers 914 of hidden units 912 by weights 910. The hidden units 912 may perform operations on one or more outputs from the hidden units 906 based, at least in part, on the weights 910. The outputs of the hidden units 912 may be provided to an output node 916 to provide an output (e.g., inference) of the neural network 900. Although one output node 916 is shown in FIG. 9, in some examples, the neural network may have multiple output nodes 916. In some examples, the output may be accompanied by a confidence level. The confidence level may be a value from, and including. 0 to 1, where a confidence level 0 indicates the neural network 900 has no confidence that the output is correct and a confidence level of 1 indicates the neural network 900 is 100% confident that the output is correct.

In some examples, inputs to the neural network 900 provided at the one or more input nodes 902 may include user-input text, user-input speech (in digitized form), log files, live capture usage data, current system settings, and/or images acquired by an ultrasound probe. In some examples, outputs provided at output node 916 may include a prediction (e.g., inference) of a user intent.

The outputs of neural network 900 may be used by an ultrasound imaging system to perform one or more tasks (e.g., change an imaging setting, retrieve patient files from a hospital server, call tech support) and/or provide one or more outputs (e.g., current software version, what anatomical view is currently being displayed).

In some examples, another processor, application, or module, such as response generator 804, may receive multiple outputs from neural network 900 and/or other neural networks that may be used to respond to the determined (e.g., predicted, inferred) user intent. For example, the response generator may receive an output indicating an anatomical feature currently being imaged by an ultrasound probe (e.g., ultrasound probe 112) of the ultrasound imaging system. The response generator may also receive an output indicating a user intent requesting measurement tools. Based on these outputs, the response generator may cause commands to be executed to provide the measurement tools used with the particular anatomy on the display.

Although a convolutional neural network has been described herein, this machine learning model has been provided only as an example, and the principles of the present disclosure are not limited to this particular model. For example, other and/or additional models may be used, such as a long short term memory model, which is often used for natural language processing.

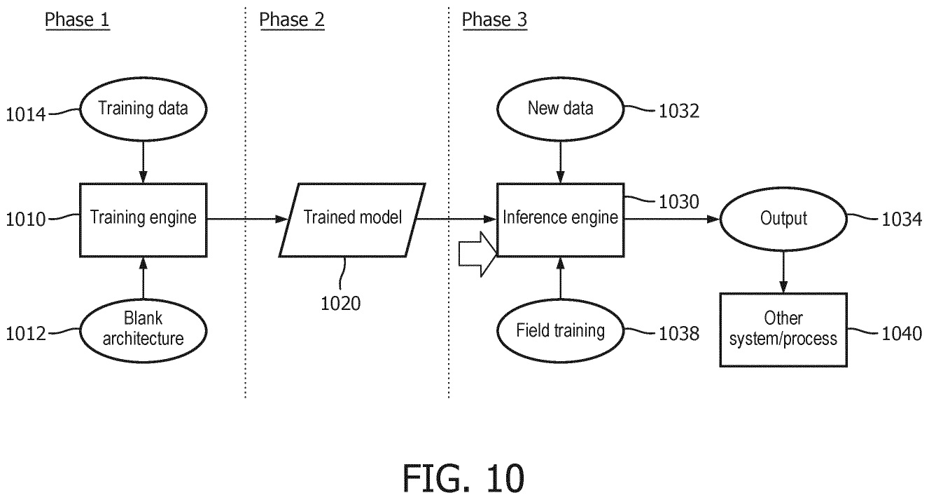

FIG. 10 shows a block diagram of a process for training and deployment of a model in accordance with the principles of the present disclosure. The process shown in FIG. 10 may be used to train a model (e.g., artificial intelligence algorithm, neural network) included in an ultrasound system, for example, a model implemented by a processor of the ultrasound system (e.g., chat bot 170). The left hand side of FIG. 10, phase 1, illustrates the training of a model. To train the model, training sets which include multiple instances of input arrays and output classifications may be presented to the training algorithm(s) of the model(s) (e.g., AlexNet training algorithm, as described by Krizhevsky. A., Sutskever. I. and Hinton. G. E. “ImageNet Classification with Deep Convolutional Neural Networks.” NIPS 2012 or its descendants). Training may involve the selection of a starting algorithm and/or network architecture 1012 and the preparation of training data 1014. The starting architecture 1012 may be a blank architecture (e.g., an architecture with defined layers and arrangement of nodes but without any previously trained weights, a defined algorithm with or without a set number of regression coefficients) or a partially trained model, such as the inception networks, which may then be further tailored for analysis of ultrasound data. The starting architecture 1012 (e.g., blank weights) and training data 1014 are provided to a training engine 1010 for training the model. Upon sufficient number of iterations (e.g., when the model performs consistently within an acceptable error), the model 1020 is said to be trained and ready for deployment, which is illustrated in the middle of FIG. 10, phase 2. The right hand side of FIG. 10, or phase 3, the trained model 1020 is applied (via inference engine 1030) for analysis of new data 1032, which is data that has not been presented to the model during the initial training (in phase 1). For example, the new data 1032 may include a question from a user during a scan of a patient (e.g., during an echocardiography exam). The trained model 1020 implemented via engine 1030 is used to analyze the unknown data in accordance with the training of the model 1020 to provide an output 1034 (e.g., a user intent). The output 1034 may then be used by the system for subsequent processes 1040 (e.g., change a setting, open a desired application). Optionally, in examples where the model 1020 is dynamically trained, field training data 1038 may be provided, which may refine the model 1020 implemented by the engine 1030.

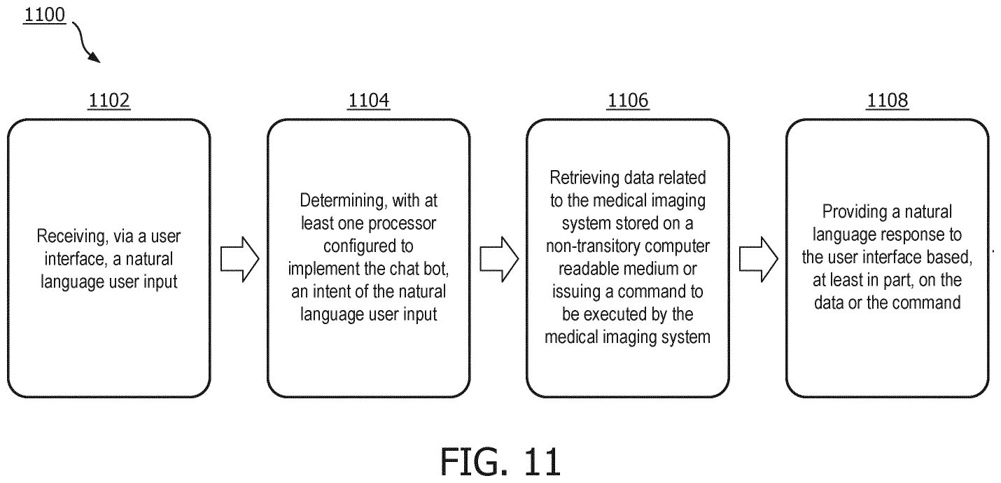

FIG. 11 is a flow chart of a method in accordance with principles of the present disclosure. The method 1100 may be performed by an imaging system, such as imaging system 100 in some examples. In some examples, some or all of the method 1100 may be performed by one or more processors, such as processor 200, included in the imaging system, such as those implementing a chat bot, such as chat bot 170 and/or chat bot 800. The method 1100 may allow a user to interact with the medical imaging system via the chat bot. The chat bot may allow the user to obtain various information (e.g., patient medical records, configuration settings of the imaging system, standard exam protocols, information on an image currently being viewed) and/or allow the user to cause the imaging system to perform various tasks (e.g., call tech support, change image acquisition settings, open an application such as a measurement toolset, etc.).

A user interface, such as user interface 124, may receive a natural language user input as indicated by block 1102. In some examples, a portion of the user interface may be included on a mobile device, such as mobile device 105. For example, the mobile device 105 may include a dialog box and text box that can receive the user input. The user input received via the mobile device may be provided to the medical imaging system. At least one processor may determine an intent of the user input as indicated by block 1104. The at least one processor may implement the chat bot in some examples. Responsive to the user intent determined at block 1104, the at least one processor may retrieve data related to the medical imaging system stored on a non-transitory computer readable medium or the at least one processor may issue a command to be executed by the medical imaging system as indicated at block 1106. In some examples, the processor that determines the intent may be different than the at least one processor that retrieves the data and/or issues a command. Based on the retrieved data and/or command, the at least one processor may provide a natural language response to the user via the user interface as indicated by block 1108. The response may be provided as text, audio, graphically, and/or other manner. In some examples, the processor that provides the response may be different than the at least one processor that determines the intent and/or the at least one processor that retrieves the data and/or issues a command.

The systems and methods disclosed herein may provide an ultrasound imaging system includes a chat bot feature that allows the user to interact with the system via text or voice to provide assistance while operating the system. The user may interact with the chat bot to resolve many types of questions involving system operation, configuration assistance, clinical assistance, training, marketing, and/or field service.

In various embodiments where components, systems and/or methods are implemented using a programmable device, such as a computer-based system or programmable logic, it should be appreciated that the above-described systems and methods can be implemented using any of various known or later developed programming languages, such as “C”. “C++”. “C #”. “Java”. “Python”, and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software and firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the invention. The functionality of one or more of the processors described herein may be incorporated into a fewer number or a single processing unit (e.g., a CPU) and may be implemented using application specific integrated circuits (ASICs) or general purpose processing circuits which are programmed responsive to executable instruction to perform the functions described herein.

Although the present system may have been described with particular reference to an ultrasound imaging system, it is also envisioned that the present system can be extended to other medical imaging systems where one or more images are obtained in a systematic manner. Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and method may be that conventional medical image systems can be easily upgraded to incorporate the features and advantages of the present systems, devices, and methods.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. A medical imaging system configured to allow a user to interact with the medical imaging system via a chat bot, the medical imaging system comprising:
a user interface configured to receive a natural language user input;
a non-transitory computer readable medium encoded with instructions to implement the chat bot and configured to store data related to the medical imaging system; and
at least one processor in communication with the non-transitory computer readable medium configured to execute the instructions to implement the chat bot, wherein the instructions cause the at least one processor to:
determine an intent of the natural language user input;
responsive to the intent, retrieve at least a portion of the data stored in the non-transitory computer readable medium or issue a command to be executed by the medical imaging system; and
provide a natural language response to the user interface based, at least in part, on the portion of the data or the command.

2. The medical imaging system of claim 1, wherein the instructions cause the at least one processor to implement a machine learning model to determine the intent of the natural language user input.

3. The medical imaging system of claim 2, wherein the machine learning model comprises a convolutional neural network.

4. The medical imaging system of claim 1, further comprising a mobile device, wherein the user interface comprises at least a portion of the mobile device.

5. The medical imaging system of claim 1, further comprising a computing system configured to store patient medical records, wherein the instructions further cause the at least one processor to retrieve at least one patient medical record from the computing system responsive to the intent and the natural language response is further based on the at least one patient medical record.

6. The medical imaging system of claim 1, wherein the instructions further cause the at least one processor to retrieve an output from a machine learning model trained to identify an anatomical feature in an image acquired by the medical imaging system responsive to the intent and the natural language response is further based on the output.

7. The medical imaging system of claim 1, wherein the natural language user input is a text input.

8. The medical imaging system of claim 1, wherein the natural language user input is an oral input.

9. The medical imaging system of claim 1, wherein the command causes the medical imaging system to change an image acquisition setting.

10. The medical imaging system of claim 1, wherein the user interface comprises a dialog box including a text box configured to allow the medical imaging system to receive the natural language user input and a send icon configured to allow the medical imaging system to provide the natural language user input to the at least one processor.

11. The medical imaging system of claim 10, wherein the user interface further comprises a cursor configured to allow a user to interact with the medical imaging system and a chat bot icon configured to cause the medical imaging system to display the dialog box responsive to the cursor hovering over the chat bot icon.

12. A method for interacting with a medical imaging system with a chat bot, the method comprising:
receiving, via a user interface, a natural language user input;
determining, with at least one processor configured to implement the chat bot, an intent of the natural language user input;
responsive to the intent:
retrieving data related to the medical imaging system stored on a non-transitory computer readable medium or issuing a command to be executed by the medical imaging system; and
providing a natural language response to the user interface based, at least in part, the data or the command.

13. The method of claim 12, wherein the user interface includes a mobile device in communication with the medical imaging system configured to receive the natural language user input.

14. The method of claim 12, further comprising retrieving an output from a machine learning model responsive to the intent wherein the natural language response is based on the output, wherein the machine learning model is configured to identify an anatomical feature included in an image acquired by the medical imaging system.

15. The method of claim 12, wherein the natural language response comprises information related to configuration settings or image acquisition settings of the medical imaging system.

16. The method of claim 12, wherein the command causes the medical imaging system to execute an application.

17. The method of claim 16, wherein the application comprises at least one of an exam protocol or a measurement tool set.

18. The method of claim 12, wherein the determining is performed by a machine learning model.

19. The method of claim 18, wherein the machine learning model comprises a convolutional neural network.

20. The method of claim 18, further comprising training the machine learning model to determine the intent of the natural language user input.
